(19) United States
(12) Patent Application Publication
(10) Pub. No.: US 2012/0052167 A1
(43) Pub. Date: Mar. 1, 2012

(54) Measuring batter dispenser and method

(76) Inventor: Rodney W. Robbins, Florence, AL (US)

(21) Appl. No.: 12/806,895

(22) Filed: Aug. 24, 2010

(51) Int. Cl. B67D 7/60; G01N 33/02; B67D 7/00

(52) U.S. Cl. 426/231; 222/386; 222/1

(57) ABSTRACT

Batter for making muffins, cupcakes, pancakes, etc. is placed in a housing with an outlet opening closed by a resilient valve. A plunger is fitted into the housing and is used to push the batter through the outlet opening. The batter ingredients can be mixed in the housing by beating or by closing the housing and shaking the ingredients. The housing has groups of markings for indicating the distances to move the plunger to dispense predetermined amounts of the batter to make food articles of predetermined sizes. Guides consisting of circular protruding ridges around the outlet opening are used to align the opening with cups in muffin pans to minimize spillage and waste.

This invention relates to measuring dispensers and methods, and particularly batter measuring dispensers and methods for dispensing batter to use in making muffins, cupcakes, pancakes, etc.

The dispensing of relatively thick liquids such as batter for making numerous small food items such as muffins, cupcakes, and pancakes, etc. long has been a problem.

When the batter is spooned or poured out onto a pancake griddle or into muffin or cupcake baking pans, there usually is spillage, and this causes waste and requires clean-up.

Also, it is difficult to dispense just the right quantity of batter for each food item, with the result that corrections of errors and uneven baking and/or serving sizes are encountered.

Some batter dispensers have been used in the past, but they suffer from deficiencies, such as excessive cost, and/or excessive spillage, and, therefore, leave much to be desired.

Therefore, it is an object of the invention to provide a batter dispenser and method in which the foregoing problems are alleviated or eliminated.

It is a specific object of the invention to provide a dispenser and method in which spillage, waste, and the need for clean-up of the spilled batter is reduced or minimized.

Another object is to provide a dispenser and method in which food items of consistent size can be prepared relatively quickly and reliably.

A further object is to provide such a device which is relatively inexpensive to manufacture, and therefore can be relatively inexpensive to buy.

The foregoing objects are met, in accordance with the invention, by the provision of a measuring batter dispenser comprising a housing with an outlet opening, a plunger to fit into the housing and push batter out of the outlet opening, a resilient valve covering the outlet opening and responsive to pressure applied to the batter by means of the plunger to allow batter to exit the opening, and markings on the housing and/or the plunger to indicate a quantity of batter dispensed corresponding to the distance moved by the plunger.

It is preferred that the plunger has a resilient seal to seal the space between the plunger and the housing.

It also is preferred that the seal on the plunger serves as a marker to align with markings on the housing to indicate a particular quantity of batter dispensed.

Preferably, the resilient cover comprises flexible flaps or fingers which serve the function of opening in response to pressure applied to the batter by the plunger, but closing automatically to stop dispensing and drips when the pressure is not applied. Therefore, one can dispense measured amounts reliably and with minimum spillage, waste and clean-up.

Also preferably, there are one or more protruding rings around the outlet opening to serve as guides to fit into batter receivers such as muffin pan cups to better guide the batter into the cups.

In one embodiment of the invention, the plunger has an easily removable cap which can be fitted onto the housing to cover the plunger entrance to facilitate mixing ingredients by shaking them in the closed housing.

Preferably, the cap has internal vanes which help mix ingredients together by shaking.

It is further preferred to provide a flexible cover or stopper for the outlet opening to seal the batter in the housing for storage, to prevent leakage through the outlet opening, especially when using the housing as a shaker, and to protect the outlet from contamination.

The dispenser can be made using relatively inexpensive plastic materials, and by relatively simple molding techniques, to provide a relatively inexpensive measuring dispenser without complex mechanisms.

The methods of using the dispenser include mixing the batter in a separate bowl or pan and pouring it into the dispenser housing, or mixing the batter in the housing.

Advantageously, the batter can be mixed in the housing by covering the housing and shaking the contents vigorously, and then inserting the plunger in the housing and using the plunger to expel the batter.

In the embodiment of the invention in which the plunger has a removable cap, the cap is removed from the plunger and fastened onto the housing to cover it, and the outlet opening is plugged to cover the housing.

Also, the batter ingredients can be mixed in the housing a whisk or fork.

The foregoing objects, features and advantages will be set forth in or apparent from the following description and drawings.

### IN THE DRAWINGS

In the drawings, FIG. 1 is a front elevation view of a batter dispenser constructed in accordance with the present invention, in a first operating position;

FIG. 2 is a front elevation view of the device shown in FIG. 1, with the device in a different operating position, and including details omitted from FIG. 1;

FIG. 3 is a right side elevation view of the device shown in FIG. 2;

FIG. 4 is rear perspective view of the device shown in FIGS. 2 and 3;

FIG. 5 is a bottom plan view of the device shown in FIGS. 1 through 4;

FIG. 6 is a partially schematic and partially broken-away cross-sectional view, taken along line 6, 8-6, 8 of FIG. 5, shown in use in filling a muffin pan;

FIG. 7 is a partially broken-away view of a portion of the device shown in FIGS. 1 through 6; and

FIG. 8 is cross-sectional view, taken along line 6, 8-6,8 of a portion of the device shown in FIG. 5;

FIG. 9 is a bottom plan view of the plunger of the device shown in FIG. 1-6, but with the guide structure 32 removed and a stopper member in place;

FIG. 10 is a partially broken-away cross-sectional view taken along line 10-10 of FIG. 9; and

FIG. 11 is a cross-sectional, partially schematic and partially broken-away view of the lower portion of the structure shown in FIG. 9;

FIG. 12 is a cross-sectional, partially schematic view of the housing bottom portion of another embodiment of the invention;

FIG. 13 is a front elevation, partially schematic, partially-exploded view of an alternative batter dispenser of the invention;

FIG. 14 is a front elevation view of the housing of the dispenser of FIG. 13, with the plunger removed and the cap of the plunger covering the housing for use as a shaker to mix batter ingredients prior to dispensing the batter; and

FIG. 15 is a front perspective view of the dispenser device of FIG. 13.

### GENERAL DESCRIPTION

A preferred batter dispenser of the present invention is indicated at 20 in FIG. 1. It consists of a housing 22 with a plunger 23 which can be moved vertically within the housing 22. The housing 22 has a bottom wall 24, and the plunger can be moved towards and away from the bottom wall 24. There is an outlet opening 63 (FIG. 5) in the bottom wall and a valve 62 to allow batter to be dispensed when the plunger is pushed downwardly against the batter in the housing.

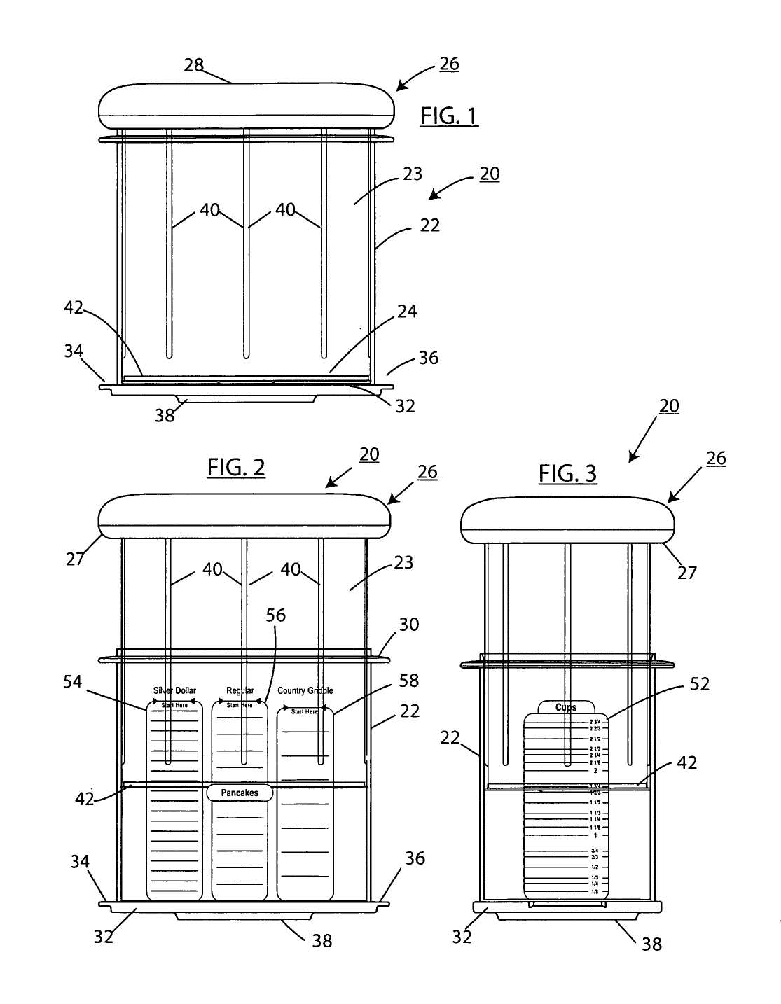

Referring particularly to FIGS. 2, 3 and 4, the housing 22 has groups of printed measurement indicia 46, 48, 50, 52, 54, 56 and 58 to assist in measuring the contents of the housing and giving measuring marks to indicate quantities of batter dispensed corresponding to variable vertical positions of the plunger 23.

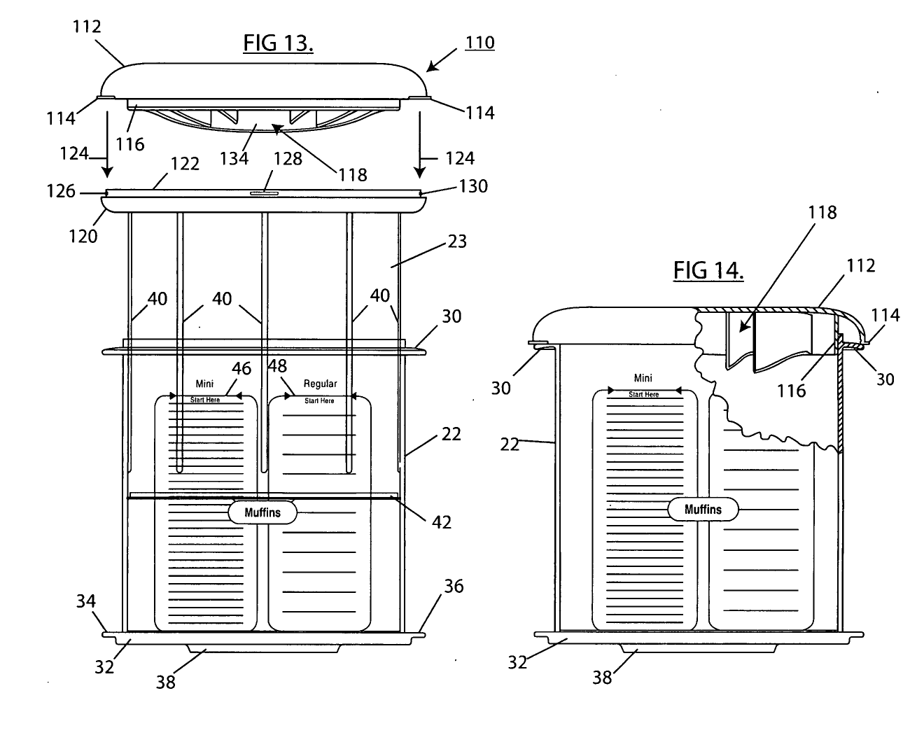

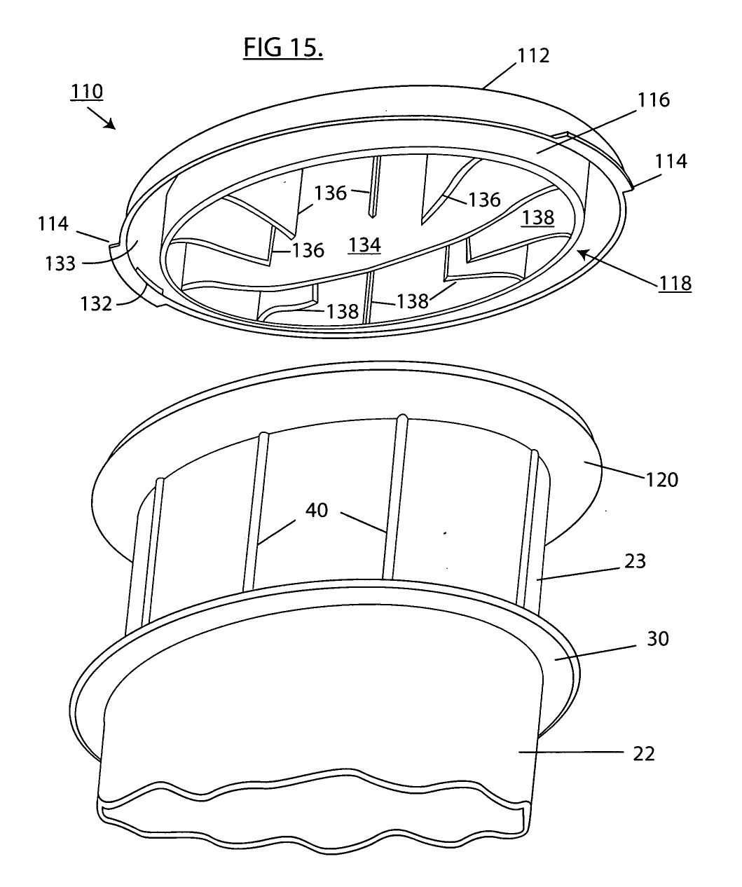

FIGS. 13, 14 and 15 show another embodiment of the invention in which the top 112 of the plunger 23 can be removed and attached to the top of the housing 22, as shown in FIG. 14, to close the top of the housing and thus facilitate its use as a shaker for mixing batter ingredients.

### DETAILED DESCRIPTION

The plunger 23 has a vertical body portion and a top portion 26 which is flat on its upper surface 28. The top portion 26 is secured to the plunger by ultra-sonic welding to a flange 27 of the plunger body.

The plunger 23 has vertical spaced-apart ribs 40 extending outwardly to contact the inner wall of the housing 22 to reduce the sliding friction between the plunger and the housing to facilitate sliding the plunger up and down in the housing.

Preferably, both the side-walls of the housing and of the plunger are elliptical in cross-sectional shape so as to maximize the housing volume while making the dispenser fit into the hand of a user more easily than if it were cylindrical.

The housing 22 has an upper lip 30 which serves as a surface to grip with the hand when pressing the plunger downwardly into the housing, and also adds strength and stiffness to the housing.

A snap-on guide 32 is removably fastened to the bottom portion of the housing 22. The guide 32 has grasping tabs 34 and 36 at opposite sides, and a downwardly-extending central guide ring 38.

Preferably, the material of which the plunger 23 is made is opaque, and the material of which the housing 22 is made is transparent so that the plunger can be seen through the housing wall.

Referring to FIG. 2, three different scales 54, 56 and 58 are provided for measuring quantities used to make small, medium, or large pancakes, respectively. The spacing between adjacent horizontal lines is largest in the group 58, smallest in the group 54 and in between those distances in the group 56.

The plunger 23 has a sealing member 42 located at the bottom edge of the plunger. The seal 42 preferably is different in appearance from the remainder of the plunger and is clearly visible through the transparent wall of the housing. For example, the seal 42 is black and the rest of the plunger is white. The distance moved by the seal 42 between two adjacent marks determines the volume of batter dispensed, and, therefore the size of the food article made by use of the device.

In using the dispenser, the housing 22 is filled with batter to a desired level, and the plunger 23 is moved to the top of the batter quantity, and then the plunger 23 is pressed downwardly by the distance between two markings on one of the scales 54, 56, or 58 to dispenser a predetermined quantity of batter through the outlet opening 63 in the housing bottom (FIG. 5).

In accordance with another feature of the invention, volume measurement scales 50 and 52 are provided at opposite sides of the housing 22. Scale 52 indicates cup measurements for the housing, and the scale 50 measures milliliters.

This feature is helpful in one of the methods of the invention in which ingredients for pancakes, muffins, cupcakes, etc. can be mixed directly in the housing. The measuring scales 50 and 52 can be used to measure the quantity of at least one component of the mixture being formed, as well as the total volume of batter in the housing.

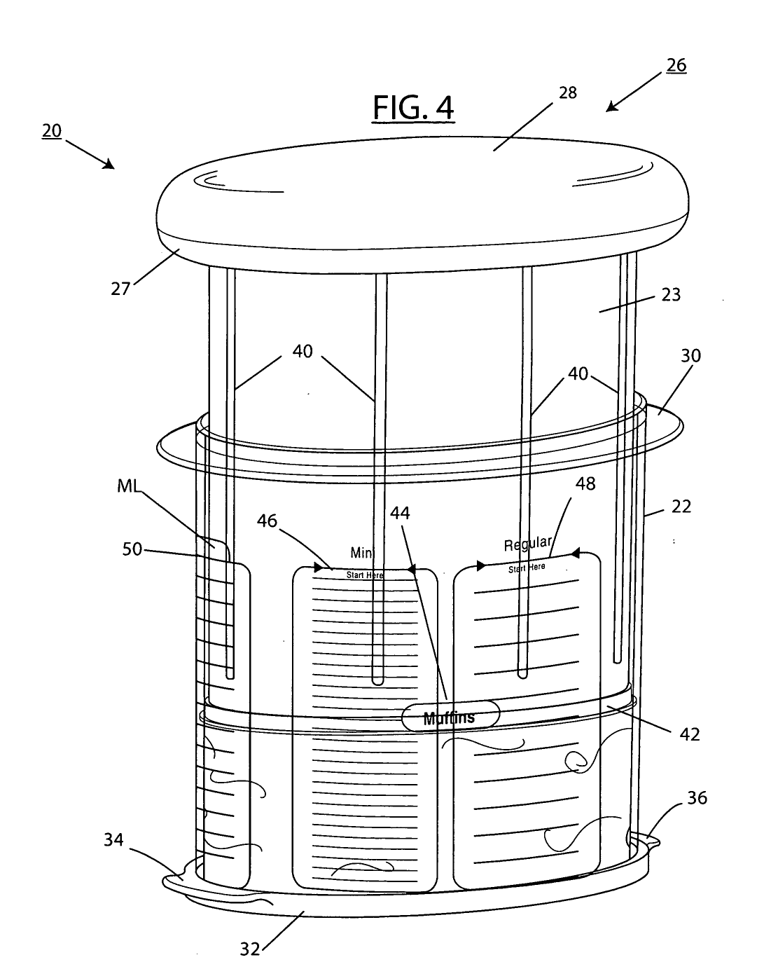

FIG. 4 shows scales 46 and 48 which are used for measuring the quantities to be dispensed to make small (“mini”) muffins or large muffins, respectively.

### Outlet Opening and Valve

FIG. 5, which is a bottom plan view of the device 20 as shown in FIGS. 1 through 4, shows the outlet opening 63 of the dispenser housing. The opening is circular and is closed by eight resilient pie-shaped silicone rubber flaps 64 which form a known valve 62. The rubber flaps are held in place between two layers of plastic material at 65. The layers of material are held together and bonded to the flaps by ultra-sonic spot welding. The pie-shaped rubber flaps 64 normally are in a flat plane and close the opening 63. However, when pressure on the batter is applied by using the plunger, the flaps 64 are bent downwardly to allow batter to be dispensed through the resulting opening. As soon as pressure is released on the plunger, the flaps return to their initial position, effectively closing the outlet opening. This is advantageous in that it greatly retards dripping and spilling of the batter.

### Locating Rings

A ring 60 surrounds the outlet opening 63 and protrudes downwardly to provide a guide for aligning the dispenser with each cup of a small muffin or cupcake-making pan.

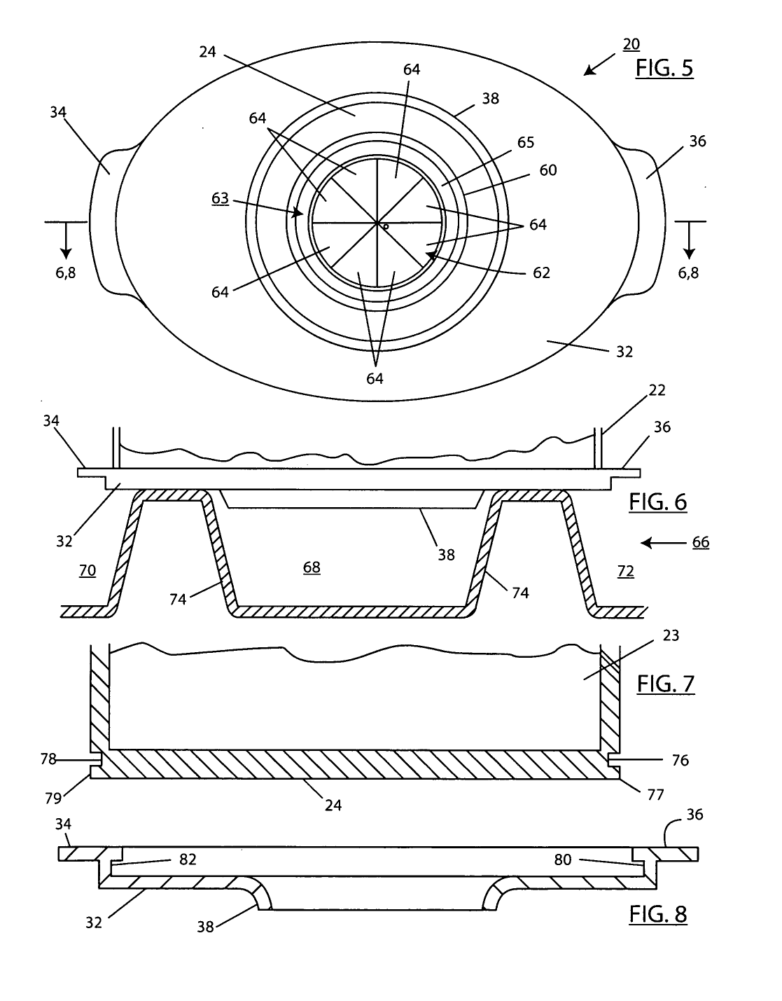

A larger ring 38 extends downwardly from the bottom of a detachable guide 32 which is used to align the dispenser with the cups of a large muffin pan, as illustrated in FIG. 6.

Referring to FIG. 6, one full cup 68 of a muffin pan indicated is shown at 74, and partial cups 70 and 72 also are shown. The ring 38 is dimensioned to fit into the cup 68 and the other cups of the pan so that when the material is dispensed downwardly out of the outlet opening 63, it unerringly enters the cup.

The guide 32 is made removable from the bottom of the housing 22 so that when it is desired to fill the small cups of a mini-muffin pan, using the guide ring 60 to guide the dispensed batter from the outlet into the mini cups of the muffin pan, the guide 32 can be removed to prevent interference which the guide 32 can cause with the mini-muffin pan.

The guide 32 is made of flexible plastic material and is held in place by the detent structure indicated in FIGS. 7 and 8. At opposite sides of the plunger 23 are two shallow, thin grooves, 76 and 78. These grooves form two short projections 77 and 79, which fit into notches 82 and 80 (FIG. 8) in the opposite sides of the guide 32. The flexible plastic of the guide 32 allows it to flex and to snap projections 77 and 79 into the grooves 80 and 82 to removably hold the guide in place. The plunger and the housing preferably are made of relatively hard, dimensionally stable, dishwasher-proof, plastic materials such as polypropylene.

An alternative guide ring structure is shown in FIG. 12. Instead of locating one of the rings 38 on a removable guide 32, both rings are formed in the bottom wall 24 of the housing.

The smaller inner ring 98 has a greater height than the outer ring 96. Thus, when the dispenser is used to fill mini-muffin cups like cups 102, 104 and 106 in a mini-muffin pan 100, the ring 98 extends into the cups to align each with the dispenser outlet, as shown in FIG. 12.

When the dispenser is used to fill “regular” sized cups as shown in FIG. 6, the taller inner ring 98 does not interfere with the outer ring 96 in its performance of its locating function.

This modification can permit the elimination of the separate guide 32.

### Outlet Plug

If the housing 22 is left full of batter for a relatively long period of time, while resting on a flat surface, the flaps 64 may bend a little under the long-term pressure of the weight of the batter, and cause a small amount of seepage of batter. Also, when mixing ingredients in the housing by shaking or beating, a secure closure for the outlet opening 63 is desirable.

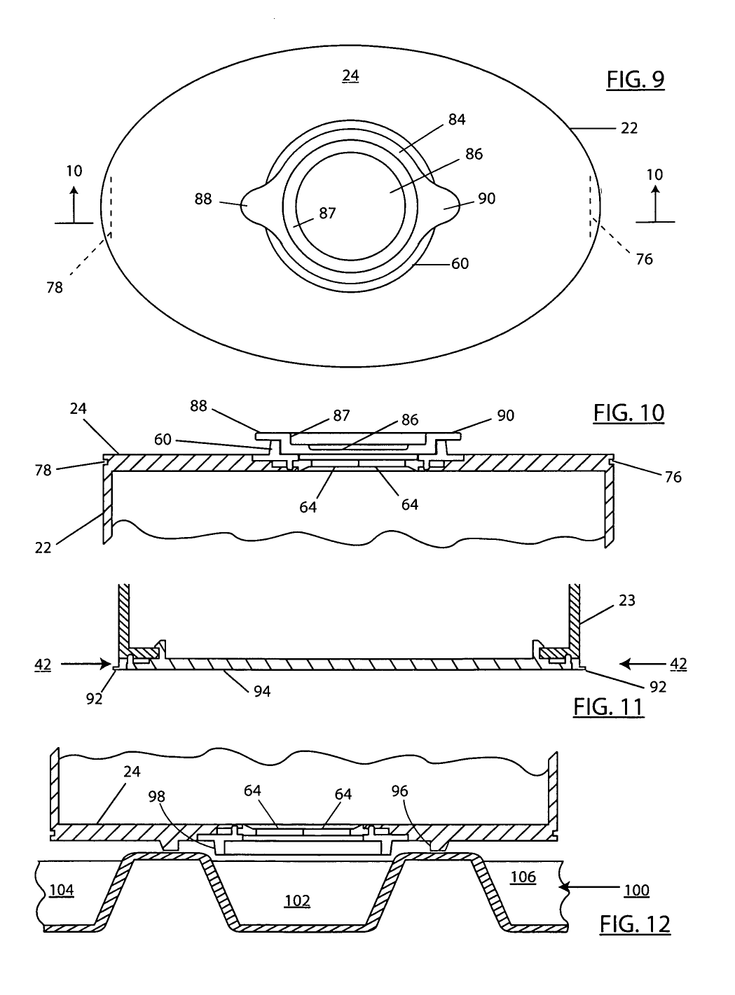

Referring now to FIGS. 9 and 10, FIG. 9 shows the bottom 24 of the housing 22, with the guide 32 removed. Instead, there is a flexible closure member or plug 84 inserted into the outlet. Specifically, the plug is inserted into the receptacle defined by the ring 60 protruding from the bottom wall 24 of housing 22. The plug 84 has a bottom wall 86 and a central recess which has an outer wall 87, as well as protruding ears 88 and 90 which are used to grasp the plug to pull it out. The plug 84 is used whenever a secure closure is needed, and in order to protect the outlet opening structure from unwanted contact with other objects and thus avoid possible damage or contamination.

### Plunger Seal

FIG. 11 is a cross-sectional view showing the details of the sealing member 42 at the bottom edge of the plunger 23. The seal has a generally L-cross-sectional shape and it is made of a silicone rubber material (preferably black or a dark color) fastened into a groove around the lower edge of the plunger 23. The seal 42 has a wiper blade 92 which extends outwardly beyond the outer surface of the plunger 22 to make wiping contact with the internal surface of the housing 22. This seal largely prevents batter from moving past the plunger, regardless of whether the device 20 is right-side-up (as in FIG. 1-4) or upside-down.

### Shaker Embodiment

Although some batter ingredients can be mixed by shaking them in the housing with the plunger 23 inserted only a short distance, mixing can be enhanced by use of the embodiment of the invention shown in FIGS. 13-15.

FIGS. 13 and 15 show a modified batter dispenser 110. It is the same as the dispenser 20 of FIGS. 1-4 except that a removable cap 112 is provided for plunger 23 instead of the fixed cap 26 of FIGS. 1-4. Also, optionally, but preferably, the cap 112 has a set 118 of internal vanes to assist in mixing batter ingredients in the housing 22.

The cap 112 has an outer rim 114 which can be grasped to snap the cap off of the plunger to use the cap to cover the top of the housing 22 for use of mixing ingredients in the housing by shaking.

Preferably, the rim 114 is provided only at the narrow ends of the cap and not on the other side areas so as to avoid unwanted removal of the cap when pulling the plunger out of the housing.

The cap has an internal wall 116 whose outer diameter is a little larger than the internal diameter of the housing 22 at its upper edge, and is slightly tapered so that it forms an interference fit with the internal surface at the upper edge of the housing when the cap 112 is fitted onto the housing as shown in FIG. 14.

The inner edge 133 (FIG. 15) of the rim of the cap 112 fits into a recess 126 (FIG. 13) in the edge of the upper flange 120 of the plunger.

The recess 126 has four horizontally-extending bumps or protrusions, only three of which are shown in the drawings at 126, 128 and 130 in FIG. 13.

FIG. 15 shows that the internal surface 133 of the rim of the cap has four indentations such as indentations 132 into which the protrusions 126, 128, 130, etc. snap when the cap 112 is pressed downwardly on the plunger top, thereby releasably securing the cap 112 to the plunger top.

The vanes 118 are shown in FIGS. 13 and 15. A large central vane 134 and plural radially-spaced vanes 136 and 138 are provided. All of the vanes are in a plane parallel to the longitudinal axis of the plunger and housing, although other arrangements of the vanes can be used, if desired.

It is believed that when the batter ingredients are thrust against the vanes, the vanes cut or break up lumps and facilitate the mixing.

When the mixing is finished, the cap 112 can be removed from the housing 22, washed, and snapped onto the top of the plunger. The vanes 118 extend downwardly into the hollow interior of the plunger.

The fastening of the cap onto the plunger by the detent mechanism described above has the further advantage of avoiding the significant cost of ultrasonic welding of the cap to the plunger, so that the construction would be beneficial even if it did not improve intra-housing ingredient mixing.

### Methods of Use

A. External Mixing

In this method, the batter is mixed externally, that is, in a separate bowl or pan, and then poured into the housing. Preferably, the batter is poured only to the top level of one of the selected marking grids. This leaves an air space at the top of the housing.

Next, the plunger is inserted a slight distance into the housing to make certain that it is properly seated, and then the device is inverted so that it rests on the flat top 28 of the plunger. The batter falls down to the bottom wall of the plunger, and the air space is moved up to the bottom wall 24 of the housing 22. Then the bottom of the housing is pushed downwardly so that the air above the batter is expelled through the outlet opening 63 until the bottom of the housing touches the batter. Then, the dispenser is inverted and is ready to dispense batter.

B. Dispensing

If pancakes are being made, the plunger is depressed to expel batter onto a griddle. Preferably, the plunger is moved between two successive horizontal lines on one of the three marking grids 54, 56 and 58 to deposit a predetermined quantity of batter on the griddle. Then, the pressure is released on the plunger and the rubber flaps 64 automatically close the outlet and cut off the flow of batter. The dispenser is moved to the next location, and the process is repeated until all the batter has been dispensed, or the griddle is full.

If regular-size muffins or cupcakes are to be made in a multi-cup muffin or cupcake pan, the guide member 32 is snapped onto the bottom of the housing, and the housing is positioned with the outer guide ring in each of the cups of the muffin pan successively, and the appropriate amount of the batter is dispensed into the cup by moving the plunger seal 42 from one mark to the next on the “regular” scale 48 (FIG. 4). Then, the other cups in the pan are filled, and the muffins are then baked in the usual way.

If mini- or small-sized muffins or cupcakes are to be made instead of those of “regular” size, the guide member 32 is removed from the housing, and the inner guide ring 60 is used to guide the batter from the outlet opening 63 into each of the smaller-sized receiving cups until they are filled. Then the smaller muffins or cupcakes are baked in the usual way.

If the alternative embodiment of the invention shown in FIG. 12 is used, it is not necessary to either remove or fasten the guide onto the housing.

C. Mixing in the Housing

In this method, the ingredients are placed directly in the housing. The first ingredient, at least, and, perhaps, others can be measured by one of the scales 50 or 52 at the sides of the housing to determine the quantity of a liquid, for example, or flour, etc. placed in the housing. Then the ingredients are beaten together with a whisk or a fork and the dispensing procedure is used as described as above to dispense the desired quantities of batter.

D. Mixing by Shaking

Ingredients also can be mixed in the housing by shaking the housing with the plunger 23 of the embodiment of FIGS. 1-12 inserted slightly into the housing.

Instead of expelling air from the space above the batter after the plunger has been inserted, with the plug 84 covering the outlet opening, the combination of the plunger and housing can be picked up and shaken to mix the ingredients. This can be faster and more satisfactory than whisking or beating with a fork.

The forcefulness of the mixing can be enhanced by providing a larger empty space in which the ingredients can move. This can be done if the amount of batter made is kept relatively small, leaving a larger air space between the ingredients and the plunger bottom.

Also, the mixing can be improved by making the cap 112 of the plunger removable, as in FIGS. 13-15, to increase the space available for shaking-type mixing. The vanes in the cap also are believed to facilitate the mixing action.

It also would be possible to make the bottom wall 94 of the plunger removable and let the ingredients move about in the internal space of the plunger.

If needed, the height of the cap 112 also can be increased to provide a greater volume usable for mixing.

E. Storage

In accordance with another feature of the invention, the dispensers provide well for the storage of left-over batter. When dispensing is finished for the day, the plug 84 can be inserted in the outlet opening, and the dispenser stored in a refrigerator. The plunger 23 and the plug 84 seal the batter in the housing with little exposure to the air, so that the batter can be used later with little loss of freshness.

It is optional that a manually-operable vent can be provided in the cover of the plunger so as to expel excess air without turning the dispenser device upside down. However, it reduces the complexity and cost of the device to use the procedure outlined above for expelling excess air from the housing prior to dispensing batter.

### Materials

As noted above, the housing and plunger preferably are made of a relatively hard, washable plastic material, such as polypropylene, and the housing 22 preferably is transparent to allow the dark seal 42 to be used as a marker. The seal 42 and the valve flaps 64 preferably are made of silicone rubber. The guide 32 is made of a flexible plastic material such as relatively thin polypropylene. The plug 84 preferably is made of “TPR” (thermo plastic rubber).

Although the invention has thus been shown and described with reference to specific embodiments, it should be noted that the invention is in no way limited to the details of the described arrangements but changes and modifications may be made without departing from the scope of the invention which is defined by the appended claims.

1. A measuring batter dispenser comprising
(a) a housing, said housing having at least one vertical side wall, a bottom wall, and a dispensing outlet in said bottom wall,
(b) a plunger dimensioned and shaped to fit and slide within said housing towards and away from said bottom wall,
(c) said plunger having at least one outer wall shaped to conform to the internal shape of said housing and move in close proximity to the inner surface of said side wall of said housing, and a plunger bottom wall,
(d) a valve closing said outlet opening, said valve being responsive to pressure from within said housing to open and allow the egress of batter therethrough, and
(f) markings on at least one of said housing and said plunger to indicate a quantity of batter dispensed corresponding to the position of said plunger.

2. A dispenser as in claim 1 in which said bottom wall has at least one protrusion extending outwardly from said bottom wall around said outlet opening to serve as a guide for positioning said outlet opening over a cup-shaped container to facilitate dispensing batter into said container.

3. A dispenser as in claim 1 including a resilient sealing member on said outer wall of said plunger for maintaining a seal between said plunger and said side wall,

4. A dispenser as in claim 2 in which said protrusion is ring-shaped, and including a flexible stopper with a protruding central portion dimensioned to be press-fitted into the enclosure formed by the inside wall of said protrusion to releasably seal said outlet opening.

5. A dispenser as in claim 2 in which said one protrusion is ring-shaped, and a second ring-shaped protrusion concentric with said one protrusion and being larger in diameter than but of shorter height than said one protrusion.

6. A dispenser as in claim 1 in which said valve comprises a plurality of resilient flaps which are temporarily bent out of the way by pressure from within said receptacle.

7. A dispenser as in claim 1 including a removable flexible plastic cover for said bottom wall of said housing, said cover having a hole with a projection around it, said hole being aligned with said outlet opening when in place, said projection being dimensioned to fit inside a receiving structure to serve as a guide for dispensing batter into said receiving structure when said cover is in place, and including a detent structure in the outside edge of said bottom wall of said housing and said cover so that said cover can be snapped onto and off of said bottom wall.

8. A dispenser as in claim 1 in which said housing has an entrance through which said plunger is inserted, said plunger of said dispenser has an external cap releasably secured thereto, said cap being dimensioned to releasably fit onto the open end of said housing to seal it when said plunger is removed, whereby shaking of the housing with said cap on it can facilitate mixing of materials in said housing,

9. A dispenser as in claim 8 in which said cap has a plurality of internal vanes positioned to intercept moving materials in said housing to facilitate mixing.

10. A dispenser as in claim 1 in which said plunger has a flat top wall capable of supporting said dispenser upside-down on a flat surface.

11. A dispenser as in claim 1 including a resilient sealing member located at the lower edge of said plunger and extending around the periphery thereof, said vertical sidewall of said housing being transparent and bearing said markings whereby said markings indicate quantities of batter proportional to the position of said sealing member relative to said bottom wall of said housing.

12. A dispenser as in claim 1 in which said housing and said plunger are approximately elliptical in cross-sectional shape.

13. A dispenser as in claim 1 in which one of the inside surface of said vertical sidewall and the outside surface of said outer wall of said plunger has protruding vertical guide ribs for easing the movement of said plunger in said housing.

14. A method of making muffins using a multi-cup muffin pan, said method comprising:
(1) providing a measuring batter dispenser having
(a) a housing, said housing having at least one vertical side wall, a bottom wall, a restricted dispensing outlet in said bottom wall,
(b) a plunger dimensioned and shaped to fit and slide within said housing towards and away from said bottom wall,
(c) said plunger having at least one outer wall shaped to conform to the internal shape of said housing and move in close proximity to the inner surface of said side wall of said housing, and a plunger bottom wall,
(d) a resilient cover over said outlet opening, said cover being responsive to pressure from within said housing to open and allow the egress of batter therethrough, and
(e) markings on at least one of said housing and said plunger to indicate a quantity of batter dispensed cones pending to the position of said plunger.
(f) at least one protrusion extending outwardly from said bottom wall around said outlet opening to serve as a guide for positioning said outlet opening over a cup-shaped container to facilitate dispensing batter into said container.
(2) placing a quantity of batter in said housing,
(3) inserting said plunger into said housing,
(4) aligning said protrusion successively with each of the cups in said pan while pressing on said plunger to dispense batter into each of said cups.

15. A method as in claim 14 in which there are at least two concentric projections from said bottom wall, two different measurement scales, and including the step of selecting one of said scales and using it to measure batter amounts dispensed into each of said cups.

16. A method as in claim 14 including selecting a scale marked on said housing and moving said plunger between two marks on said scale for each dispensing operation.

17. A method of dispensing batter from a dispenser to make a plurality of items from a single batch of batter, comprising
(1) Providing a measuring batter dispenser having
(a) a housing, said housing having at least one vertical side wall, a bottom wall, a restricted dispensing outlet in said bottom wall,
(b) a plunger dimensioned and shaped to fit and slide within said housing towards and away from said bottom wall,
(c) said plunger having at least one outer wall shaped to conform to the internal shape of said housing and move in close proximity to the inner surface of said side wall of said housing, and a plunger bottom wall,
(d) a resilient valve at outlet opening, said valve being responsive to pressure from within said housing to open and allow the egress of batter therethrough, and
(e) markings on at least one of said housing and said plunger to indicate a quantity of batter dispensed corresponding to the position of said plunger.
(2) Locating a quantity of batter in said housing.
(3) Moving said plunger to push said batter out of said housing through said outlet by the distance between two marks in said markings.

18. A method as in claim 16 in which said markings include a plurality of different scales for different sizes and types of said food items, and including the step of selecting one of said scales for the desired size and type of food item to be made.

19. A method as in claim 16 in which said locating step comprises mixing the ingredients of said batter in said housing when said housing is closed.

20. A method as in claim 19 in which said plunger of said dispenser has an external cap releasably secured thereto, said cap being dimensioned to releasably fit onto the open end of said housing to seal it when said plunger is removed, whereby shaking of the housing with said cap on it can facilitate mixing of materials in said housing, and in which said cap has a plurality of internal vanes positioned to intercept moving materials in said housing to facilitate mixing and including the steps of removing said cap from said plunger and onto said entrance of said housing, and shaking said housing to mix the ingredients.
